(19) United States
(12) Patent Application Publication
(10) Pub. No.: US 2002/0029113 A1
(43) Pub. Date: Mar. 7, 2002

(54) Method and system for predicting splice variant from DNA chip expression data

(76) Inventors: Yixin Wang, San Diego, CA (US); Gang Hu, Ann Arbor, MI (US)

Correspondence Address: PILLSBURY WINTHROP LLP, 1600 TYSONS BOULEVARD, MCLEAN, VA, 22102, US

(21) Appl. No.: 09/934,083

(22) Filed: Aug. 22, 2001

Related U.S. Application Data
(60) Provisional application No. 60/226,680, filed on Aug. 22, 2000.

(51) Int. Cl. G06F 19/00; G01N 33/48; G01N 33/50

(52) U.S. Cl. 702/20

(57) ABSTRACT

A system and method predict alternative splicing transcripts using DNA chip expression data as a primary data source. The system and method may perform prediction of alternative splicing of pre-messenger RNA that may be used, for example, for regulating eukaryotic gene expression.

### CROSS REFERENCE TO RELATED APPLICATION

This application claims priority from U.S. Provisional Patent Application Serial No. 60/226,680, filed Aug. 22, 2000, the content of which is hereby incorporated by reference herein in its entirety.

### BACKGROUND OF THE INVENTION

1. Field of the Invention

The invention relates to a data processing system and method of use for analyzing gene expression data using a computer algorithm.

2. Description of Related Art

Cells regulate the expression of their genes in response to environmental changes. Normally this regulation is beneficial to the cell, protecting it from starvation or injury; however errors in this regulation can lead to serious diseases ranging from cancer to heart disease. Measuring the differential expression of genes from various stages of an organism's development in different tissues and organisms subjected to different stresses provides information instrumental in understanding the relationships between genes and their functions. Studying gene regulation is useful for both assaying drugs and as a source of new molecular targets, assuming the regulatory network controlling a given gene is well understood. As such, changes in gene expression patterns can be used to assay drug efficacy throughout the drug discovery process.

One assay that takes advantage of the existing level of sequence information, and that is complementary to sequence and genetic analysis, is gene expression profiling. Expression profiling can be carried out by one of a number of different technologies, such as commercially or privately manufactured gene chips, which typically measure the expression level of thousands of genes simultaneously using an array of oligonucleotides bound to a silicon surface. These arrays are hybridized under stringent conditions with a complex sample representing mRNAs expressed in the test cell or tissue. Target sequences hybridize to immobilized oligonucleotides and are typically detected via fluorescent signals.

Relative intensity levels of the fluorescent signals indicate relative gene expression in a given sample obtained from a source subjected to a particular condition. As a sample source is subjected to a variety of conditions, a given gene will display a profile under these conditions. The results from these expression profiling technologies are quantitative and highly parallel, thereby allowing an accurate snapshot to be made of the workings of the cell in a particular state.

Since thousands of hybridization reactions may occur in a single array, expression profiling assays generate huge data sets that are not amenable to simple analysis. To maximize the use of such data, efforts are underway to develop algorithms interpreting and interconnecting results for different genes under different conditions.

Alternative splicing is an essential biological process that generates multiple different transcripts from the same precursor mRNA. Alternative splicing is an important regulatory mechanism for high eukaryotic gene expression (Elliott, D. J. 2000. Splicing and the single cell. Histol. Histopathol. 15: 239-249; Gelfand, M. S., Dubchak, I., Dralyuk, I., and Zorn, M. 1999. ASDB: database of alternatively spliced genes. Nucleic Acids Res. 27: 301-302; Lopez, A. J. 1998. Alternative splicing of pre-mRNA: developmental consequences and mechanisms of regulation. Annu. Rev. Genet. 32: 279-305; and Smith, C. W., Patton, J. G., and Nadal-Ginard, B. 1989. Alternative splicing in the control of gene expression. Annu. Rev. Genet. 23: 527-577). It is estimated that upwards of 35% of human genes undergo alternative splicing during development, cellular differentiation and other cellular processes (Mironov, A. A., Fickett, J. W., and Gelfand, M. S. 1999. Frequent alternative splicing of human genes. Genome Res. 9: 1288-1293; Wolfsberg, T. G., and Landsman, D. 1997. A comparison of expressed sequence tags (ESTs) to human genomic sequences. Nucleic Acids Res. 25: 1626-1632.). However, alternative splicing is tightly regulated with temporal and tissue specific patterns.

Aberrant splicing of precursor transcripts has been associated with various human diseases (Crook, R., Verkkoniemi, A., Perez-Tur, J., Mehta, N., Baker, M., Houlden, H., Farrer, M., Hutton, M., Lincoln, S., Hardy, J., Gwinn, K., Somer, M., Paetau, A., Kalimo, H., Ylikoski, R., Poyhonen, M., Kucera, S., and Haltia, M. 1998. A variant of Alzheimer's disease with spastic paraparesis and unusual plaques due to deletion of exon 9 of presenilin 1. Nat. Med. 4: 452-455; Mottes, J. R. and Iverson, L. E. 1995. Tissue-specific alternative splicing of hybrid Shaker/lacZ genes correlates with kinetic differences in Shaker K+ currents in vivo. Neuron 14: 613-623; Weissensteiner, T. 1998. Prostate cancer cells show a nearly 100-fold increase in the expression of the longer of two alternatively spliced mRNAs of the prostate-specific membrane antigen. Nucleic Acids Res. 26: 687; Wilson, C. A., Payton, M. N., Elliott, G. S., Buaas, F. W., Cajulis, E. E., Grosshans, D., Ramos, L., Reese, D. M., Slamon, D. J., and Calzone, F. J. 1997. Differential subcellular localization, expression and biological toxicity of BRCA1 and the splice variant BRCA1-delta11 b. Oncogene 14: 1-16; Jiang, Z. H., and Wu, J. Y. 1999. Alternative splicing and programmed cell death. Proc. Soc. Exp. Biol. Med. 220: 64-72). As a result, analysis of tissue- and disease- specific splice variations may provide important insight into the mechanism(s) of normal cellular as well as disease processes.

However, it is difficult to learn the tissue-specific pattern of alternative splicing of tens of thousands of genes using traditional molecular biology approaches. Moreover, the current knowledge of splice variants in publicly accessible databases is fragmented. Recent efforts have been made to collect that information from annotated databases, e.g., SWISSPROT, and expressed sequence tag (EST) databases (Wolfsberg et al. 1997; Gelfand et al. 1999). It has also been shown that by using a sequence clustering procedure, a rich source of splice variants can be identified from EST sequences (Mironov et al. 1999).

Moreover, recent technological advances, such as high-density oligonucleotide arrays, allow biologists to study gene expression at a genome scale (Chee, M., Yang, R., Hubbell, E., Berno, A., Huang, X. C., Stem, D., Winkler, J., Lockhart, D. J., Morris, M. S., and Fodor, S. P. A. 1996. Accessing genetic information with high-density DNA arrays. Science 274: 610-614; Lipshutz, R. J., Fodor, S. P. A., Gingeras, T. R., and Lockhart, D. J. 1999. High density synthetic oligonucleotide arrays. Nat. Genet. Suppl. 21: 20-24). The Affymetrix™ DNA chip technology is based on hybridization of labeled RNA probes with gene specific oligonucleotide arrays on the surface of a glass chip. By detecting the intensity of hybridizing probes on the chip, one can analyze the expression level of thousands of genes simultaneously. Since each gene is represented by a number of pairs of oligonucleotide probes spanning the 3′ region, DNA chips also offer a unique opportunity to assess 3′ splice variants of the gene.

### SUMMARY OF THE INVENTION

In view of the above, the exemplary embodiment of the present invention is directed to a system and method, or one or more components thereof, for predicting alternative splicing transcripts using DNA chip expression data as a primary data source.

### BRIEF DESCRIPTION OF THE DRAWINGS

The exemplary embodiment of the present invention is further described in the detailed description which follows, by reference to a noted plurality of drawings, by way of non-limiting exemplary embodiment of the present invention, in which like reference numerals represent similar parts throughout the several views of the drawings and wherein:

FIG. 1 is a block diagram of a gene expression profiling data analysis system;

FIG. 2 is a flow chart illustrating a method for prediction of alternative splice variants in accordance with the exemplary embodiment of the invention;

FIG. 3 is a flow chart illustrating a Sample Preparation and Hybridization Phase of the method for prediction of alternative splice variants illustrated in FIG. 2;

FIG. 4 is a flow chart illustrating a Data Preprocessing Phase of the method for prediction of alternative splice variants illustrated in FIG. 2;

FIG. 5 is a flow chart illustrating a SPLICE Algorithm Phase of the method for prediction of alternative splice variants illustrated in FIG. 2; and

FIG. 6 is a flow chart illustrating a NEIGHBORHOOD Algorithm Phase of the method for prediction of alternative splice variants illustrated in FIG. 2.

### DETAILED DESCRIPTION OF THE EXEMPLARY EMBODIMENT

For purposes of clarification, and to facilitate an understanding of the present invention and the exemplary embodiment disclosed herein, a number of terms used herein are defined as follows. The term “expression profiling” refers to a process by which gene expression techniques are used to measure and compare expression levels of certain gene transcripts or levels of certain gene products, such as polypeptides or proteins, in a cell-derived sample in relation to the levels of the same transcripts or proteins from a different sample, or from the same sample measured at a different time point. An “enzyme” is a protein that catalyzes biochemical reactions. A “protein molecule” is one or several polypetide chains of amino acids. The term “gene” refers to a sequence of nucleotides specifying a particular polypeptide chain. The term “gene product” refers to one or several polypeptide chains of amino acids translated from RNA transcribed from a gene.

“RNA” stands for Ribonucleic acid. The term “mRNA” refers to messenger Ribonucleic acid. An “mRNA” or “messenger RNA” is an RNA molecule synthesized from a DNA template—by the enzyme RNA polymerase. An mRNA functions as a template for the assembly of a polypeptide chain, a process known as translation. An “RNA Polymerase” is an enzyme that synthesizes RNA by using DNA as a template. The term “transcription” refers to a process by which an RNA molecule is synthesized by the enzyme RNA polymerase using DNA as a template.

The exemplary embodiment provides a system and method for predicting alternative splicing transcripts using DNA chip expression data as a primary data source. Such DNA chip expression data may be provided using any high density, oligonucleotide probe micro-array, for example, an oligonucleotide array of 1600 rat genes. In such an example, each gene on the chip may be represented by, for example, twenty pairs of perfect match and mismatch oligonucleotide probes. Using such oligonucleotide probe micro-arrays, chip hybridization data may be collected from one or more types of tissues, for example, different rat tissues such as bladder, eye, heart, kidney, large intestine, small intestine, liver, pancreas, placenta, testis and skeletal muscle. To predict potential tissue-specific splice variants, algorithms are used to process and normalize the initial chip hybridization data at the oligonucleotide probe level. A first data processing algorithm, hereinafter referred to as the “SPLICE” algorithm may be used to process raw hybridization signals directly collected from chip scanning images. In the SPLICE algorithm, tissue-specific expression data generated by each oligonucleotide probe may be normalized, transformed, filtered and compared. Subsequently, the SPLICE algorithms may output an initial prediction of which oligonucleotide probes have been determined as candidate probes for hitting potential alternative splicing regions.

To improve the accuracy of this initial determination, a second data processing algorithm, hereinafter referred to as the “NEIGHBORHOOD” algorithm may be used to process the output of the “SPLICE” algorithm and relate that to the location of the probes on the gene. The rationale for using the NEIGHBORHOOD algorithm lies in an assumption that an alternative splicing region may, and most likely will, span multiple probes in an array because of the size of the alternative splicing region. Therefore, if the probes that neighbor an identified candidate probe also generate data that may indicate the presence of a splicing region, the determination of the candidate probe is to some extent confirmed by corroborating data generated by the neighbor probes. However, if probes neighboring an identified candidate probe have generated data that does not indicate the presence of a splicing region, the initially identified candidate probe may be eliminated as a candidate. This elimination is based on the assumption that an alternative splicing region may, and most likely will, span multiple probe locations in an array because of its size. Therefore, any initially determined candidate probe will be discounted unless its status is confirmed by corroborating data generated by neighbor probes.

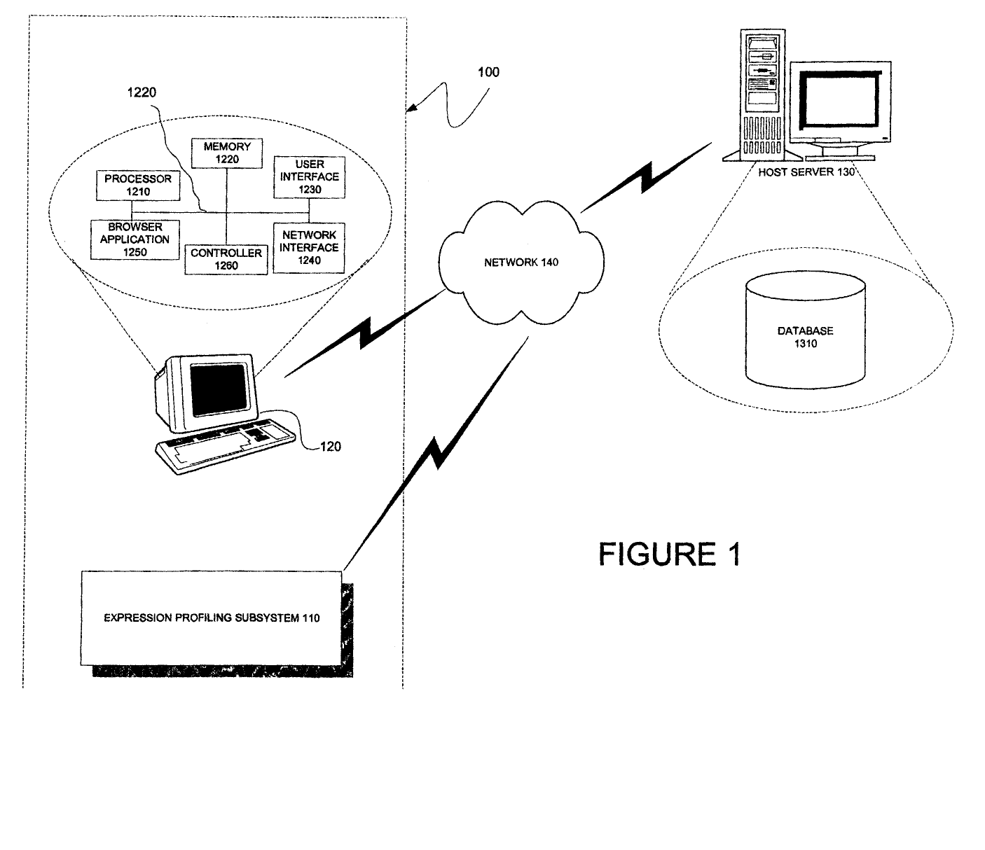

FIG. 1 illustrates functional block diagram of a system for analyzing gene expression data 100 designed in accordance with the exemplary embodiment of the invention. An expression profiling subsystem 110 is provided, which is coupled to a user terminal 120. The user terminal 120 may comprise, among other elements, a processor 1210, a memory 1220, a user interface 1230, a network interface 1240, a browser application 1250, the software of which is stored in the memory 1220 and running on the processor 1210. The user interface 1230 may be implemented in any standard or other interface for facilitating human interaction with and control of terminal 120, including, for example, a keyboard, a mouse, a monitor, speakers, etc. The user terminal 120 may be coupled to a host server 130 via a communication network 140, e.g., a public or private network such as a wide area network, a local area network, an intranet or the Internet. Host server 130 may incorporate and provide access to a database 1310.

The network 140 may provide access to the host server 130 that may be operated and maintained by an entity to provide information that may be downloaded to the terminal 120 and may relate to gene profiling data. It is foreseeable, the user may access such a host server to download information, for example, gene profiling data in the database 1310 for use by the processor 1210. Moreover, it is foreseeable that the network interface 1240 may be used in conjunction with the bus 1220 and network 140 to upload information from the terminal 120 to the server 130 to augment information within the database 1310.

The controller 1260 operates to control operation of the other elements 1210-1250 of the terminal 120. It should be appreciated that the controller 1260 may be implemented with the processor 1210, for example, in a central processing unit, or other similar device. The processor 1210 works with the controller 1260 to control operation of the other elements 1220-1250. In cooperation with the controller 1260, the processor 1210 may fetch instructions from memory 1220 and decode them, which may cause the processor 1210 to transfer data to or from memory 1220 or to work in combination with the user interface 1230 (for example, to input or output information), the expression profiling subsystem 110 (for example, to input data or output instructions from or to the expression profiling subsystem 110), the network interface 1240 (for example, to upload/download information to/from the host server 130), etc.

The memory 1220 may be implemented using static or dynamic RAM and/or ROM. However, the memory 1220 can also be implemented using a floppy disk and disk drive, a writable optical disk and disk drive, a hard drive, flash memory or the like.

The user interface 1230 may include, for example, a display, key board and mouse. Moreover, the user interface 1230 may include a speaker and microphone, not shown, for outputting and inputting information to and from a user. The user interface 1230 may operate in conjunction with the processor 1210 and controller 1260 to allow a user to interact with software programs stored in the memory 1220 and used by the processor 1210 as well as to allow the user to interact with software programs run on the host server 130 via the network 140.

The network interface 1240 operates in conjunction with the control/communication/data bus 1220 to provide communication between the terminal 120 and the network 140, which may be a publicly or privately accessible network, e.g., the Internet. Thus, the signal lines or links that couple the terminal 120 to the server 130 may be a public switched telephone network, a local or wide area network, an intranet, the Internet, a wireless transmission channel, any other distributing network, or the like.

The browser application 1250 may be used by the processor 1210 to access the information in the database 1310 via the network 140.

It should be understood that each of the elements 1210-1260 can be implemented, for example, as portions of a suitably programmed general purpose or specific purpose computer.

The expression profiling subsystem 110 may comprise, among other things, any high density, oligonucleotide probe micro-array, for example, an Affymetrix® GeneChip. Such arrays provide efficient access to genetic information. Within such a probe array, a set of oligonucleotide probes to be synthesized is defined, based on its ability to hybridize to the target loci or genes of interest. The array generates, from control and treatment sets of cell-derived samples, respective sets of gene expression data representing a direction and a magnitude of regulation of each one of a high number of different nucleic acid sequences.

More specifically, by way of example, a sample of cells may be analyzed using an expression profiling array, such as an Affymetrix GeneChip™ probe array for, for example, the human genome, which is capable of detecting over 65,000 sequences for that genome. Affymetrix™ provides a GeneChip™ fluidics station that automates the hybridization of nucleic acid targets to a probe array cartridge, and thus controls the delivery of reagents and the timing and temperature for hybridization. Each fluidics station can independently process four probe arrays at a given time.

Accordingly, each target may be prepared from a set of cell dishes or tissue samples by isolation of RNA over a course of time. The treatment of those cells may be emulated by adding, for example, serum thereto. At predetermined intervals, a small amount of the fluid is removed, and the cells are put in a quiescent state to stop the reaction time. Accordingly, a large set of targets, having a predetermined amount of liquid (e.g., 0.5 ml each) is produced. The GeneChip™ fluidics station may then hybridize each target, i.e., extract all the RNA and label the RNA by adding a chemical tag to each molecule, and control the delivery of the resulting liquid to the probe arrays to facilitate the obtaining of expression information regarding the mRNAs. The amount of mRNA is then ascertained based upon the signal strength of the reading given by the probe at the appropriate location corresponding to that sequence or sequence segment.

The nucleic acid to be analyzed—the target—may be isolated, amplified and labeled with a fluorescent reporter group. The labeled target may then be incubated with the array using the fluidics station. After the hybridization reaction is complete, the array may be inserted into the scanner, where patterns of hybridization are detected. The hybridization data may be collected as light emitted from the fluorescent reporter groups already incorporated into the target, which is now bound to the probe array. Probes that perfectly match the target generally produce stronger signals than those that have mismatches. Since the sequence and position of each probe on the array are known, by complementarity, the identity of the target nucleic acid applied to the probe array can be determined.

The operation and cooperation of the expression profiling subsystem 110, the terminal 120, and the host server 130 together with the user interface 1230 and browser application 1250, allows a user to operate the system for analyzing gene expression data 100. The expression profiling subsystem 10 obtains the expression profiling data and stores that data in an organized fashion in database 1310 via the network 140. Under the direction of the controller 1160, the terminal 120 communicates with database 1310 using the network interface 1240 through the network 140 and host server 130.

The host server 130 is provided with, among other elements, an analysis application for performing certain analysis associated with expression profiling and managing the data acquired from the expression profiling. A database server software component is also provided on the host server 130 for handling and acting on database queries and responses.

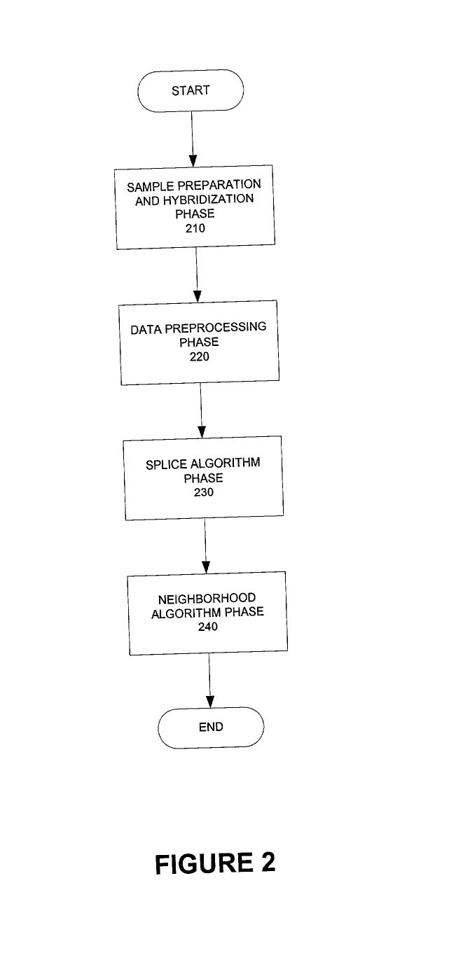

As shown, in FIG. 2, a method for prediction of alternative splice variants according to the exemplary embodiment of the invention includes four main phases: a Sample Preparation and Hybridization Phase 210, a Data Preprocessing Phase 220, a SPLICE Algorithm Phase 230 and a NEIGHBORHOOD Algorithm Phase 240.

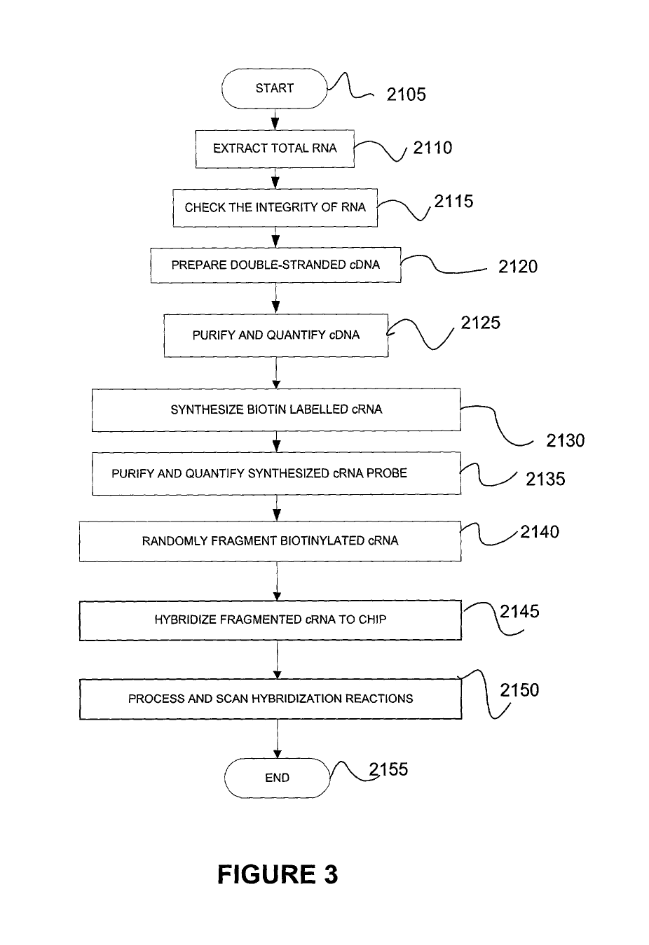

As shown in FIG. 3, the Sample Preparation and Hybridization Phase 210 begins at 2105 and control proceeds to 2110. At 2110, the total RNA from a set of tissue samples is extracted, for example, RNAs of normal rat tissue samples: bladder, eye, heart, kidney, large intestine, small intestine, liver, pancreas, placenta, testis and skeletal muscle may be extracted using TRIZOL™ reagent (Life Technologies™ Inc., Gaitherburg, Md.). Control then proceeds to 2115, at which transcript integrity is monitored using, e.g., denaturing agorose gel electrophoresis.

Control then proceeds to 2120, at which double-stranded cDNA are prepared, for example, from 15 μg of total RNA using a modified oligo-dT primer with a 5' T7 RNA polymerase promoter sequence and the Superscript Choice System for cDNA Synthesis (Life Technologies™ Inc., Gaithersburg, Md.). Control then proceeds to 2125, at which the cDNA is purified and quantified. This may be performed using a phenol-chloroform extraction and ethanol precipitation. Control then proceeds to 2130, at which the biotin labeled cRNA is synthesized. This may be performed using one-half of the cDNA reaction (0.5- 1.0 μg) as a template in an in vitro transcription reaction (BioArray™ High Yield Kit, ENZO™, Inc.) containing T7 RNA polymerase, a mixture of unlabeled ATP, CTP, GTP, and UTP, and biotin-11-CTP and biotin-16-UTP. Control then proceeds to 2135, at which the resulting cRNA may be purified, for example, on an affinity resin (RNeasy™, Qiagen™), and quantified using, for example, the convention that 1 O.D. 260 corresponds to 40 μg/ml of RNA. Subsequently, control proceeds to 2140, at which, a quantity, e.g., 15 μg, of biotinylated cRNA is randomly fragmented to an average size of, for example, 50 nucleotides, e.g., by incubating at 94° C. for 35 minutes in 40 mM TRIS-acetate, pH 8.1, 100 mM potassium acetate, and 30 mM magnesium acetate. Control then proceeds to 2145, at which the fragmented cRNA may be hybridized, for example, hybridized for 16 hours at 45° C. on a custom Affymetrix GeneChip™ containing probes for 1600 individual rat genes in a solution containing 100 mM MES, 1 M [Na+], 20 mM EDTA, 0.01% TWEEN 20, 50 pM of Control Oligonucleotide B2 (Afffymetrix™, Inc.), 0.1 mg/ml of sonicated herring sperm DNA, and 0.5 mg/ml BSA. Each hybridization may include, for example, a mixture of four bacterial biotinylated-RNA transcripts (BioB, BioC, BioD, and cre) spiked at 1.5, 5, 25, and 100 pM, respectively. Control then proceeds to 2150, at which the hybridization reactions are processed and scanned according to standard Affymetrix™ protocols. After chip scanning, control then proceeds to 2155, at which the Sample Preparation and Hybridization Phase ends and control proceeds to the Data Preprocessing Phase 220.

It should be appreciated that the Sample Preparation and Hybridization Phase may be performed multiple times for different samples of various tissues and/or chips to provide a large data set and to minimize the effect of localized errors.

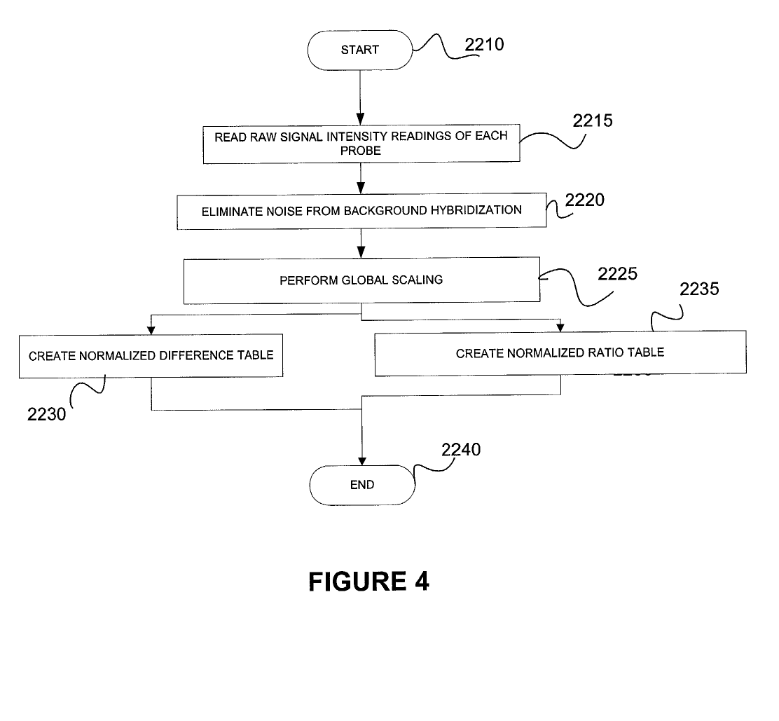

As shown in FIG. 4, the Data Preprocessing Phase 220 begins at 2210 and control proceeds to 2215. At 2215, the raw signal intensity readings of each probe on the chip are extracted, for example, from the .CEL files generated by the Affymetrix software. This extraction may involve various operations on the .CEL files, including extracting chip coordinate information from the probe sets to determine what genetic material is contained on the chip and their location. Control then proceeds to 2220, at which noise from background hybridization is eliminated by, for example, using the average of the lowest 2% of the probe signals as background noise and subtracting that background noise level from each probe signal on the chip. Control then proceeds to 2225, at which global scaling is performed for the data from each chip to further normalize signals collected from the different chips. Control then proceeds to 2230, at which a normalized difference table is created by subtracting each mismatch signal from its corresponding perfect match signal within the normalized and scaled data. Simultaneously, at 2235, a normalized ratio table is generated by dividing the perfect match and mismatch signals of each probe pair.

Control then proceeds to 2240, at which the Data Preprocessing Phase ends and control proceeds to the SPLICE Algorithm Phase 230.

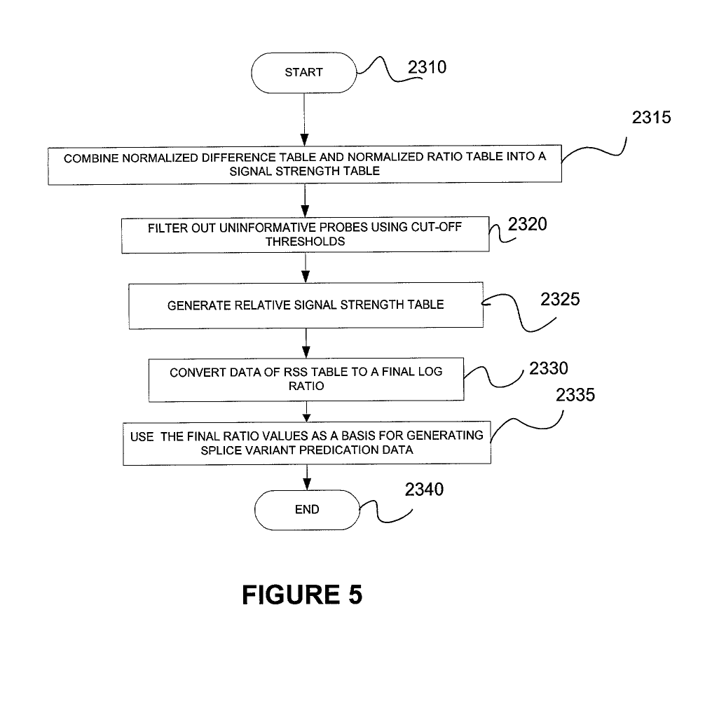

During the SPLICE Algorithm Phase, candidate probes recognizing potential tissue specific splice variants are predicted by the SPLICE algorithm. The SPLICE algorithm may filter out the oligonucleotide probe set and attempts to detect tissue specific splice variants. As shown in FIG. 5, the SPLICE Algorithm Phase 230 begins at 2310 and control proceeds to 2315. At 2315, the normalized difference table and the normalized ratio table are combined into a signal strength table (CSS) by assigning a default difference value (0) for each probe pair with a ratio equal to or less than a minimum ratio cutoff, e.g., 1.2.

Control then proceeds to 2320, at which several cut-off thresholds may be used to filter out uninformative probes. That is, to simplify the calculations for formulating the RSS table and reduce outliner effects, several cut-off thresholds may be used in the normalization. Min_Diff and Max_Diff are the minimum difference and maximum difference cut-off, the default may be 20 and 5000, respectively. Signals that either above or below the cutoffs are replaced by the cutoff values. After applying the Min and Max cutoffs on the CSS table, the average difference of each probe set in each tissue [AvgD(I, x)] can be calculated, as well as the average difference of each probe across different tissues [AvgDi]. Non-informative probe threshold (NIPT) functions to take away the probe pairs with no or very low expression in all the tissues collected, the default may be set at AvgDi>30. To consider the situations that there is no or extremely low expression of a gene in a particular tissue, a non-informative tissue type threshold (NITT) is used to eliminate those tissues from the prediction process for that particular probe set.

The default value may be set to AvgD(I, x)>30. For cases in which a few probes give strong hybridization signals in comparison with the rest of the probe set, a single probe threshold (SPT) may be used to differentiate the signals from the otherwise non-informative probe set. The default value for SPT may be set at 200. After obtaining tissue specific relative signal strength for each probe, the relative expression of the gene at each probe region can be compared among different tissues.

Control then proceeds to 2325, at which a tissue-specific Relative Signal Strength Table (RSS) is generated by normalizing the expression level across tissues in the normalized and thresholded CSS table data. The formula for the conversion is:

RSS(i, x)=D(i, x)/AvgD(I, x)

where RSS(i, x) represents the relative signal strength value of probe pair i within probe set I in tissue X. D(i, x) is the difference value of probe pair i in tissue X from the CSS table. AvgD(I,x) is the trimmed mean difference of probe set I in tissue X. Control then proceeds to 2330, at which the data of the RSS table is converted to a final log ratio to further amplify the difference of relative probe signals across tissues. Capturing and amplifying the difference among tissues further converts the RSS value for each probe pair to a final ratio (or log final ratio), which reflect the differential relative expression of the probe among those tissues. The formula for the conversion is:

FR(i, x)=Ln(RSS(i, x)/Avg_{—}RSS(i, (n−x)))

where FR(i, x) is the final log ratio of probe i in tissue X. RSS(i, x) represents the relative signal strength value of probe pair i in tissue X. Avg_RSS(i, (n−x)) is the average RSS value of probe pair i in all tissues except tissue X.

Control then proceeds to 2335, at which the FR value may be used as a basis for generating splice variant prediction data. Probes with absolute FR values greater than 1n(R) in a particular tissue may be selected as candidate probes from that tissue. R is the selection ratio, the default of which may be set at 10.

Control then proceeds to 2340, at which the SPLICE Algorithm Phase ends and control may proceed to the NEIGHBORHOOD Algorithm Phase 240.

To improve the accuracy of the initial prediction provided by the SPLICE Algorithm Phase, control proceeds to the NEIGHBORHOOD Algorithm Phase 240. The NEIGHBORHOOD algorithm measures the relative position of probes on the gene and generates a final prediction of splice variants on the genome scale.

Use of the NEIGHBORHOOD algorithm is based on the assumption that most alternatively spliced regions on a gene are large enough to contain two or more consecutive probes. Accordingly, a set of oligonucleotide probes, for example, 20, for each gene fragment may be aligned to correlate with the physical location of those probes matching 5′ to 3′ orientation of the gene. The NEIGHBORHOOD algorithm assesses the relative locations of the probes selected by the SPLICE algorithm as potential locations of splice variants so that single probes or non-consecutive probes can be filtered out. The NEIGHBORHOOD algorithm uses a probes/gene ratio, i.e., the number of candidate probes per gene or probe set (the default may be set to three) and a probes/cluster ratio, i.e., the number of consecutive probes per cluster or splicing neighborhood (the default may be set to two).

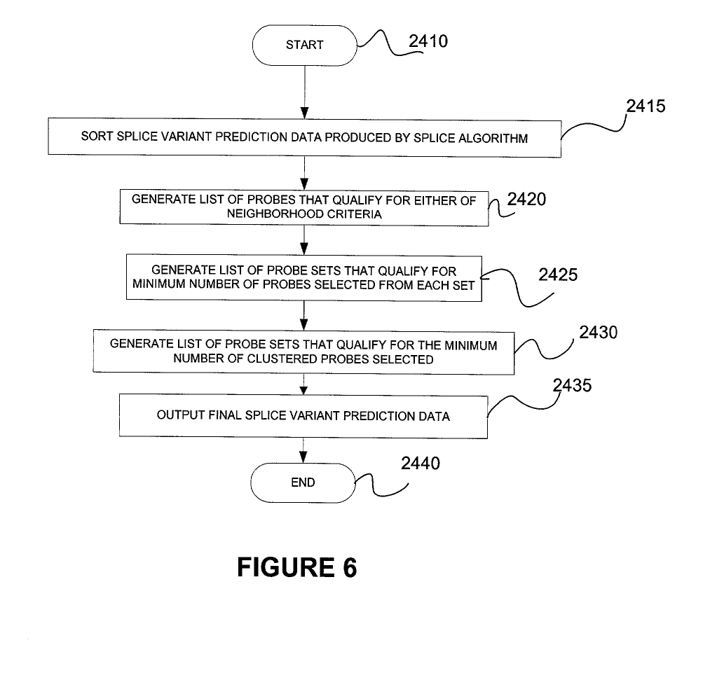

As shown in FIG. 6, the NEIGHBORHOOD Algorithm Phase 240 begins at 2410 and control proceeds to 2415. At 2415, the splice variant prediction data generated at 2335 is sorted to prioritize the data. Control then proceeds to 2420, at which a first list is generated, which is a list of probes that qualify for either of the neighborhood selection criteria. This list is generated based on the relative location of probes on the gene, which is correlated with the location of probes on the chip. Control then proceeds to 2425, at which a second list is generated, which is a list of probe sets that qualify for the minimum number of probes selected from each set. This list is also generated based on the relative location of probes on the gene. Control then proceeds to 2430, at which a third list is generated, which is a list of probe sets that qualify for the minimum number of clustered probes selected. This list is also generated based on the relative location of probes on the gene. The list generated at 2430 may be used for the final splice variant prediction data. The list generated in 2420 may used to provide detailed probe location information. Control then proceeds to 2435, at which the splice variant prediction data is output.

Control then proceeds to 2440, at which the NEIGHBORHOOD Algorithm Phase and the splice prediction method illustrated in FIG. 2 ends.

As previously described, each probe set on a high density oligonucleotide array consists of different oligonucleotide probes complementary to the 3′ sequences within a target gene (Lockhart, D. J., Dong, H., Byrne, M. C., Follettie, M. T., Gallo, M. V., Chee, M. S., Mittmann, M., Wang, C., Kobayashi, M., Horton, H., and Brown, E. L. 1996, Expression monitoring by hybridization to high-density oligonucleotide arrays. Nat. Biotechnol. 14: 1675-1680). The average hybridization signals of a probe set reflect the overall abundance of the target mRNA. In addition, the hybridization signal from an individual probe correlates with the expression level of the transcript complementary to that particular probe. This relationship establishes the basis of using an array of oligonucleotide probes or DNA chip to differentiate alternatively spliced transcripts.

Experiments were performed in a laboratory setting to confirm that the above-described method for predicting splice variants was effective. During these experiments, the SPLICE and NEIGHBORHOOD algorithms were tested and the heuristics of those algorithms used in the prediction were improved. In the experiments, expression data of three rat tissues was collected using a custom designed Affymetrix®rat1600 chip. The chip contained an array of 1600 rat genes and ESTs. Twenty pairs of oligonucleotide probes were selected against the 3′ sequence of each target gene. Separate but identical probe labeling and chip hybridization experiments were performed using RNA samples extracted from normal rat heart, liver and skeletal muscle. To optimize the prediction algorithms, SPLICE and NEIGHBORHOOD methods were applied to the data set at different selection strengths.

Table 1 illustrates the splice variant prediction provided by the SPLICE and NEIGHBORHOOD Algorithm Phases from three rat tissues: heart, liver and skeletal muscle tissues. Table 1(A) illustrates the splice variant prediction from a triplicate control experiment. Independent RNA labeling and chip hybridization experiments were performed as triplicate for each tissue sample. Potential splice variants were predicted from each set of triplicate data using the SPLICE algorithm alone or in combination with the NEIGHBORHOOD algorithm. Subsequently, the total number of predictions from each tissue set was calculated.

Table 1(B) illustrates the splice variant prediction from three different rat tissues. To generate the data set of three different tissues, the mean CSS value of each tissue triplicate was calculated and appended into the same table. Similar splice variant predictions were performed using the combined data set from the three tissues.

The triplicate data set illustrated in Table 1(A) on the same tissue (heart, liver, skeletal muscle) was used as a negative control to tune the parameters in the SPLICE algorithm. By increasing the selection ratio (R) from 5 to 10 fold, the number of total genes selected from all three tissues using both algorithms (SP+NB) decreased from 20 to 9(Table 1(A)). However, further increasing of R did not effectively decrease the number of prediction, suggesting that number may represent the residual background noise in the data set.

In comparison with predictions from triplicate data set of the same tissues, the algorithms generated a much greater number of candidates from the data set of different tissues illustrated in Table 1(B). Since consistent conditions were applied during the experiment, this difference may represent tissue specific expression of alternative transcripts. To eliminate background noise and retain prediction sensitivity, R=10 was used as default selection strength value for making the following predictions. Other heuristics in the algorithms also affect the prediction result but in a minor way as compared to the selection ratio.

The default values listed in the explanation of the Data Preprocessing Phase above have generated consistent prediction results.

The splice variant prediction method described above is based on relative gene expression among different tissues at probe level. It is reasonable to assume that the more tissue types included in the data set the more potential splice variant can be detected. To confirm this hypothesis and further test the prediction method and system, further experiments were performed in which, Rat1600 chip expression data was collected from ten different rat tissues, including bladder, eye, heart, kidney, large intestine, small intestine, liver, pancreas, placenta and testis. By using a selection ratio (R) of ten, the SPLICE algorithm used in combination with the NEIGHBORHOOD algorithm predicted that a total of 268 genes may have alternative transcripts and the alternative splicing affect 1218 probes. Table 2 illustrates the splice variant prediction from the ten normal rat tissues. Total RNA of the tissues was extracted, labeled and hybridized to the Rat1600 chip using standard and identical procedures. Individual feature hybridization data were collected and normalized as described in the above. The number of predictions for each tissue type was calculated separately. The selection ratio (R) was set at 10 and other default cut-off value were applied.

As expected, the numbers were significantly higher in comparison with those from the triplicate tissue experiment in Table 1(A). It also shows that potential splice variants can be detected across all tissues analyzed. The result also indicates that there is a higher chance of detecting potential splice variants in pancreas, testis, placenta and liver tissues.

Table 3 shows a list of top candidate splice variants predicted from the ten normal rat tissues illustrated in Table 2. The top candidate splice variants were selected by both algorithms and ranked by a scoring matrix used in the NEIGHBORHOOD algorithm. In Table 3, the identity of each probe set is represented in the first column as genebank accession number; “Tissue” indicates the tissue type from where the splice variant was predicted; “FR” is log final ratio, “+” and “−” value represent present and absent of expression, respectively; “X” and “Y” represent the chip location of individual probes detecting a splice region; “probes/cluster” and “probes/gene” indicates the number of consecutive probes in each splicing neighborhood and the total number of predicted probes from each gene, respectively. For the scoring, probes/cluster was set equal or greater than two, probes/cluster was set equal to probes/gene and the absolute value of FR was set greater than 2.5.

Based upon the expression data provided by the above-described three tissue experiments, it was predicted that about 4.5% (69 out of 1600) of the genes on the chip contain potential splice variants. Since this is just a prediction from expression data of three tissues, it was likely an underestimate of the actual number of splice variants. The expression data from ten rat tissues predicted a significantly greater number of potential splice variants (17%). However, some recent studies based on EST clustering data suggest that upwards of 35% of mammalian genes contain alternative splicing (Mironov et al. 1999; Wolfsberg et al. 1997). Nevertheless, the number of human genes containing splice variants involving 3′ exons is believed to be much lower (Mironov et al. 1999).

Accordingly, probe selection for the current DNA chips is biased toward the 3′ sequence of a gene. Therefore, it may only be possible to assess the status of alternative splicing in the 3′ region (usually ˜600 bp upstream of polyA signal) of the gene. However, it should be appreciated the operations described above can be easily applied to expression data generated by 5′ probes when that becomes available. To effectively analyze alternative splicing across the whole gene, probes need to be selected so that they spread a greater length of the transcript.

While this invention has been described in conjunction with the specific embodiment outlined above, it is evident that many alternatives, modifications and variations will be apparent to those skilled in the art. Accordingly, the exemplary embodiment of the invention, as set forth above, is intended to be illustrative, not limiting. Various changes may be made without departing from the spirit and scope of the invention.

For example, the operations performed during the SPLICE Algorithm Phase may be practiced with other operations that are different and/or independent from the other phases identified in this disclosure. Therefore, it should be appreciated that the operations of the SPLICE algorithm may be used with other data generating and preprocessing operations. Moreover, it should be appreciated that the operations performed in the Sample Preparation and Hybridization Phase 210, Data Preprocessing Phase 220 and SPLICE Algorithm Phase 230 may be practiced without the NEIGHBORHOOD Algorithm Phase 240 because the operations of the NEIGHBORHOOD Algorithm Phase 240 may be unnecessary or disadvantageous.

Additionally, it should be appreciated that the operations and algorithms described above can be applied to data obtained from an expression profiling subsystem that uses oligonucleotide based Microarray technology.

Further, it should be appreciated that the accuracy of systems and methods for splice variant prediction depend on several factors. The most important is data consistency or reproducibility. Sample variation is a major contributor of error rate (data not shown) and is usually caused by difference in tissue preparation and RNA extraction protocols. To ensure consistency in sample preparation, a highly repeatable tissue and RNA extraction procedure should be utilized. RNA labeling and chip hybridization process can also introduce variations, though the data generated from the triplicate experiments suggest that variations from independent labeling and hybridization processes can be minimized when follow strict protocols. To minimize the variations, the same lot of DNA chips should be used for splice variant prediction. To further reduce data inconsistency, dual color experiments may prove to be a powerful approach to assess subtle transcript differences in DNA chip experiment (Hacia, J. G., Brody, L. C., Chee, M. S., Fodor, S. P. A., and Collins, F. S. 1996. Detection of heterozygous mutations in BRCAI using high density oligonucleotide arrays and two-color fluorescence analysis. Nat. Genet. 14: 441-447; Chee et al. 1996); however, a control tissue sample with known splice variant status may be needed.

The size of the data set also may contribute to the accuracy of splice variant prediction. Theoretically, the more tissue types, or samples from different developmental stages, included in the raw data generated by the expression profiling subsystem 110, the more splice variants that can be detected. This relationship should be confirmed by the significant increase of predicted potential splice variants in ten rat tissues in comparison with those from three tissues.

Additionally, better chip design may dramatically improve the accuracy of splice variant prediction and increase the usefulness of the technique. Background noise encountered during the above-described experiments may be partly attributed to physical defects on the chip, such as scratches or debris from manufacturing. By introducing duplicate or triplicate probes on a chip and using probe scrambling techniques, the data variations from such defects may be nearly eliminated. Smart probe selection based on EST cluster information may also greatly improve the efficiency of splice variant detection. Ideally, the selected oligonucleotide probes should be derived from as many different alternative transcripts as possible and evenly distributed across the overall length of the transcript. The ability to design such probes depends heavily on a comprehensive EST cluster database with large tissue specific transcripts information. Expansion of current public and private EST projects should eventually reach this goal.

Lastly, a robust probe selection algorithm may help to design a next generation of DNA chips including tissue specific splice variant detection chips.

We claim:

1. A system for predicting alternative splicing transcripts using DNA chip expression data, the system comprising:
an expression profiling subsystem configured to provide DNA chip expression data;
a processor, coupled to the expression profiling subsystem configured to analyze the DNA chip expression data,
a network interface, coupled to both the processor and the expression profiling subsystem that is configured to provide access to data related to gene profiling.

2. The system of claim 1, wherein the network interface is coupled to a network that is coupled to a host server that stored the data related to gene profiling.

3. The system of claim 1, further comprising a memory coupled to the processor configured to store operating instructions for the processor.

4. The system of claim 3, further comprising a user interface coupled to the processor and the network interface, the user interface being configured to provide the capability of interacting with software programs stored in the memory and used by the processor.

5. The system of claim 4, wherein the user interface is also configured to provide access to data related to gene profiling via the network interface, which is coupled to a network that is coupled to a host server that stores the data related to gene profiling.

6. The system of claim 1, further comprising a controller coupled to the processor and the network interface and that controls operation of the processor and the network interface and cooperation of the processor, network interface and expression profiling subsystem.

7. The system of claim 1, wherein the expression profiling subsystem comprises at least one high density, oligonucleotide probe micro-array.

8. The system of claim 7, wherein the micro-array includes a set of oligonucleotide probes that generate, from control and treatment sets of cell-derived samples, respective sets of gene expression data representing a direction and a magnitude of regulation of nucleic acid sequences.

9. The system of claim 1, wherein the expression profiling subsystem obtains the DNA chip expression data and stores that data in an organized fashion in a host server coupled to the processor via the network interface.

10. The system of claim 9, further comprising a controller coupled to the processor and the network interface and that controls operation of the processor and the network interface and cooperation of the processor, network interface and expression profiling subsystem, wherein the controller controls the cooperation between the processor and the host server.

11. The system of claim 10, wherein the host server is provided with an analysis application for performing analysis associated with expression profiling and managing the data acquired from the DNA chip expression data.

12. A method for predicting alternative splicing transcripts using DNA chip expression data, the method comprising:
performing test sample preparation and hybridization for a set of tissue samples during which hybridization reactions of the set of tissue samples are scanned;
preprocessing data resulting from the scanned hybridization reactions; and
performing a first splice variant prediction to produce first splice variant prediction data.

13. The method of claim 12, further comprising performing a second splice variant prediction to produce second splice-variant prediction data.

14. The method of claim 12, wherein sample preparation and hybridization comprises:
extracting total RNA from the set of tissue samples;
preparing double-stranded cDNA from the extracted total RNA;
performing phenol-chloroform extraction and ethanol precipitation on the double-stranded cDNA to produce a cDNA reaction;
using one-half of the cDNA reaction as a template in an in vitro transcription reaction to produce cRNA;
purifying and quantifying the cRNA;
randomly fragmenting the cRNA;
hybridizing the randomly fragmented cRNA; and
scanning the results of hybridization.

15. The method of claim 12, wherein preprocessing data resulting from the scanned hybridization reaction comprises:
extracting raw signal intensity readings of each probe on the DNA chip in the data resulting from the scanned hybridization reaction;
normalizing the extracted raw signal intensity readings by removing noise resulting from background hybridization from the extracted raw signal intensity readings;
performing global scaling on the normalized raw signal intensity readings;
generating a normalized difference table by subtracting each mismatch signal from its corresponding perfect match signal within the normalized and scaled intensity readings; and
generating a normalized ratio table by dividing the perfect match and mismatch signals of each probe pair within the normalized and scaled intensity readings.

16. The method of claim 12, wherein performing a first splice variant prediction to produce first splice variant prediction data comprises:
combining a normalized difference table and a normalized ratio table produced by the preprocessing step to generate a signal strength table;
filtering out data in the signal strength table that corresponds to uninformative probes using at least one cut-off threshold;
calculating the average difference of each probe set in each tissue sample;
calculating the average difference of each probe across different tissue samples;
calculating tissue-specific relative signal strength data by normalizing the expression level across tissues in the normalized and thresholded signal strength data; and
convert the tissue-specific relative signal strength data to a final log ratio.

17. The method of claim 13, wherein performing a second splice variant prediction to produce second splice-variant prediction data comprises sorting splice variant prediction data generated by performing a first splice variant prediction to prioritize the data.
